(19) United States
(12) Patent Application Publication
(10) Pub. No.: US 2023/0128907 A1
(43) Pub. Date: Apr. 27, 2023

(54) PARIS POLYPHYLLA SAPONIN EXTRACT WITH ACTIVITY AGAINST MULTIDRUG-RESISTANT BACTERIA AND PREPARATION AND USE THEREOF

(71) Applicants: Jia WAN, Xi'an (CN); Chunchun KONG, Xi'an (CN); Shunjun DING, Xi'an (CN); Jingyi LI, Xi'an (CN); Lei TIAN, Xi'an (CN)

(72) Inventors: Jia WAN, Xi'an (CN); Chunchun KONG, Xi'an (CN); Shunjun DING, Xi'an (CN); Jingyi LI, Xi'an (CN); Lei TIAN, Xi'an (CN)

(73) Assignee: XI'AN KANGYUANSHENG BIOMEDICAL TECHNOLOGY CO., LTD., Xi'an (CN)

(21) Appl. No.: 17/942,941

(22) Filed: Sep. 12, 2022

(30) Foreign Application Priority Data
Oct. 22, 2021 (CN) 202111236303.X

(51) Int. Cl. A61K 36/896; A61K 31/7048; A61P 31/04

(52) U.S. Cl. CPC A61K 36/896; A61K 31/7048; A61P 31/04; A61K 2236/333; A61K 2236/55

(57) ABSTRACT

A method of preparing a Paris polyphylla saponin extract includes: (1) adding Paris polyphylla saponin in 60-90% ethanol to obtain a mixture, heating the mixture under reflux for 2-4 hours; (2) concentrating the mixture to ¼ volume, centrifuging the mixture to obtain a supernatant A and a precipitate A, adding the precipitate A to 60-80% ethanol to obtain a supernatant B and a precipitate B, washing the precipitate B with water and petroleum ether and drying; and (3) combining the supernatant A and the supernatant B, mixing with a macroporous adsorption resin, loading to a chromatography column, eluting the chromatography column with water and 80-90% ethanol, collecting an ethanol eluent, concentrating the ethanol eluent to obtain an eluent extract, and combining the eluent extract and the precipitate B and drying to obtain the Paris polyphylla saponin extract.

This application claims priority to Chinese Patent Application No. 202111236303.X, filed on Oct. 22, 2021, which is incorporated by reference for all purposes as if fully set forth herein.

### TECHNICAL FIELD

The invention relates to a Paris polyphylla saponin extract with activity against multidrug-resistant bacteria and preparation and use thereof.

### BACKGROUND TECHNIQUE

Paris polyphylla saponin is one of the main raw materials of Chinese medicines. At present, there are many methods for preparing Paris polyphylla saponin extract, but the separation and purification methods are complicated and the yield is low. There is a need for an improved method of preparing Paris polyphylla saponin extract,

### SUMMARY OF THE INVENTION

In one embodiment, the present application provides a method of preparing a Paris polyphylla saponin extract. The method includes: (1) adding Paris polyphylla saponin in 60-90% ethanol to obtain a mixture, heating the mixture under reflux for 2-4 hours; (2) concentrating the mixture to ¼ volume, centrifuging the mixture to obtain a supernatant A and a precipitate A, adding the precipitate A to 60-80% ethanol to obtain a supernatant B and a precipitate B, washing the precipitate B with water and petroleum ether and drying; and (3) combining the supernatant A and the supernatant B, mixing with a macroporous adsorption resin, loading to a chromatography column, eluting the chromatography column with water and 80-90% ethanol, collecting an ethanol eluent, concentrating the ethanol eluent to obtain an eluent extract, and combining the eluent extract and the precipitate B and drying to obtain the Paris polyphylla saponin extract.

In another embodiment, in step (1), a weight ratio of the Paris polyphylla saponin and 60-90% ethanol is 1: 8-24.

In another embodiment, in step (2), a weight ratio of the precipitate A and 60-80% ethanol is 1: 4-10.

In another embodiment, the Paris polyphylla saponin extract includes 0.12%-0.14% of polyphyllin II, 0.06%-0.11% of polyphyllin VI and 0.11%-0.15% of polyphyllin VII.

### DESCRIPTION OF DRAWINGS

FIG. 1 shows the content of polyphyllin II, polyphyllin VI and polyphyllin VII in the Paris polyphylla saponin extract of Examples 5-8: (a) Example 5, (b) Example 6, (c) Example 7, and (d) Example 8.

### DETAILED DESCRIPTION

In order to make those skilled in the art better understand the solutions of the present invention, the technical solutions in the embodiments of the present invention will be clearly and completely described below with reference to the accompanying drawings in the embodiments of the present invention. Obviously, the described embodiments are only part of the present invention. Based on the embodiments of the present invention, all other embodiments that can be obtained by a person of ordinary skill in the art without creative efforts shall fall within the protection scope of the present invention.

Below in conjunction with accompanying drawing, the present invention is described in further detail:

### 1. Preparation of Paris polyphylla Saponin Extract

### Example 1

One kg of Paris polyphylla saponin was added to 8 kg of 60% ethanol, soaked overnight, and heated under reflux twice, 2 hours each time. The mixture was concentrated to ¼ volume, filtered to obtain a supernatant 1 and a precipitate 1. The precipitate 1 was added to 4 times the amount of 60% ethanol, stirred, and centrifuged to obtain a supernatant 2 and a precipitate 2. The precipitate 2 was washed with 100 mL of water and petroleum ether sequentially, and dried. The supernatant 1 and supernatant were combined, and mixed with a macroporous adsorption resin (AB-8) in a weight ratio of 1:10, and loaded to a chromatography column. The chromatography column was eluted with 5 times the volume of water and 4 time the volume of 80% ethanol at a speed of 1 BV/h. The ethanol eluent was collected, concentrated, combined with the precipitate 2, and dried to 17.53 g of Paris polyphylla saponin extract.

### Example 2

One kg of Paris polyphylla saponin was added to 16 kg of 80% ethanol, soaked overnight, and heated under reflux twice, 3 hours first time and 2 hours second time. The mixture was concentrated to ¼ volume, filtered to obtain a supernatant 1 and a precipitate 1. The precipitate 1 was added to 7 times the amount of 70% ethanol, stirred, and centrifuged to obtain a supernatant 2 and a precipitate 2. The precipitate 2 was washed with 100 mL of water and petroleum ether sequentially, and dried. The supernatant 1 and supernatant were combined, and mixed with a macroporous adsorption resin (AB-8) in a weight ratio of 1:7, and loaded to a chromatography column. The chromatography column was eluted with 7 times the volume of water and 6 time the volume of 80% ethanol at a speed of 2 BV/h. The ethanol eluent was collected, concentrated, combined with the precipitate 2, and dried to 18.04 g of Paris polyphylla saponin extract.

### Example 3

One kg of Paris polyphylla saponin was added to 24 kg of 60-95% ethanol, soaked overnight, and heated under reflux twice, 2 hours each time. The mixture was concentrated to ¼ volume, filtered to obtain a supernatant 1 and a precipitate 1. The precipitate 1 was added to 10 times the amount of 80% ethanol, stirred, and centrifuged to obtain a supernatant 2 and a precipitate 2. The precipitate 2 was washed with 100 mL of water and petroleum ether sequentially, and dried. The supernatant 1 and supernatant were combined, and mixed with a macroporous adsorption resin (AB-8) in a weight ratio of 1:10, and loaded to a chromatography column. The chromatography column was eluted with 10 times the volume of water and 8 time the volume of 80% ethanol at a speed of 3 BV/h. The ethanol eluent was collected, concentrated, combined with the precipitate 2, and dried to 18.65 g of Paris polyphylla saponin extract.

### Example 4

One kg of Paris polyphylla saponin was added to 8 kg of 60% ethanol, soaked overnight, and heated under reflux three times, 2 hours each time. The mixture was concentrated to ¼ volume, filtered to obtain a supernatant 1 and a precipitate 1. The precipitate 1 was added to 7 times the amount of 80% ethanol, stirred, and centrifuged to obtain a supernatant 2 and a precipitate 2. The precipitate 2 was washed with 100 mL of water and petroleum ether sequentially, and dried. The supernatant 1 and supernatant were combined, and mixed with a macroporous adsorption resin (AB-8) in a weight ratio of 1:7, and loaded to a chromatography column. The chromatography column was eluted with 5 times the volume of water and 8 time the volume of 95% ethanol at a speed of 1 BV/h. The ethanol eluent was collected, concentrated, combined with the precipitate 2, and dried to 18.92 g of Paris polyphylla saponin extract.

### Example 5

One kg of Paris polyphylla saponin was added to 8 kg of 95% ethanol, soaked overnight, and heated under reflux twice, 3 hours first time and 2 hours second time. The mixture was concentrated to ¼ volume, filtered to obtain a supernatant 1 and a precipitate 1. The precipitate 1 was added to 7 times the amount of 80% ethanol, stirred, and centrifuged to obtain a supernatant 2 and a precipitate 2. The precipitate 2 was washed with 100 mL of water and petroleum ether sequentially, and dried. The supernatant 1 and supernatant were combined, and mixed with a macroporous adsorption resin (AB-8) in a weight ratio of 1:5, and loaded to a chromatography column. The chromatography column was eluted with 5 times the volume of water and 8 time the volume of 80% ethanol at a speed of 2 BV/h. The ethanol eluent was collected, concentrated, combined with the precipitate 2, and dried to 19.83 g of Paris polyphylla saponin extract.

### Example 6

One kg of Paris polyphylla saponin was added to 16 kg of 95% ethanol, soaked overnight, and heated under reflux twice, 3 hours first time and 2 hours second time. The mixture was concentrated to ¼ volume, filtered to obtain a supernatant 1 and a precipitate 1. The precipitate 1 was added to 7 times the amount of 80% ethanol, stirred, and centrifuged to obtain a supernatant 2 and a precipitate 2. The precipitate 2 was washed with 100 mL of water and petroleum ether sequentially, and dried. The supernatant 1 and supernatant were combined, and mixed with a macroporous adsorption resin (LX-T5) in a weight ratio of 1:5, and loaded to a chromatography column. The chromatography column was eluted with 5 times the volume of water and 8 time the volume of 80% ethanol at a speed of 2 BV/h. The ethanol eluent was collected, concentrated, combined with the precipitate 2, and dried to 18.21 g of Paris polyphylla saponin extract.

### Example 7

One kg of Paris polyphylla saponin was added to 8 kg of 95% ethanol, soaked overnight, and heated under reflux twice, 3 hours first time and 2 hours second time. The mixture was concentrated to ¼ volume, filtered to obtain a supernatant 1 and a precipitate 1. The precipitate 1 was added to 7 times the amount of 80% ethanol, stirred, and centrifuged to obtain a supernatant 2 and a precipitate 2. The precipitate 2 was washed with 100 mL of water and petroleum ether sequentially, and dried. The supernatant 1 and supernatant were combined, and mixed with a macroporous adsorption resin (D101) in a weight ratio of 1:5, and loaded to a chromatography column. The chromatography column was eluted with 5 times the volume of water and 8 time the volume of 80% ethanol at a speed of 2 BV/h. The ethanol eluent was collected, concentrated, combined with the precipitate 2, and dried to 17.37 g of Paris polyphylla saponin extract.

### Example 8

One kg of Paris polyphylla saponin was added to 24 kg of 95% ethanol, soaked overnight, and heated under reflux twice, 3 hours first time and 2 hours second time. The mixture was concentrated to ¼ volume, filtered to obtain a supernatant 1 and a precipitate 1. The precipitate 1 was added to 7 times the amount of 80% ethanol, stirred, and centrifuged to obtain a supernatant 2 and a precipitate 2. The precipitate 2 was washed with 100 mL of water and petroleum ether sequentially, and dried. The supernatant 1 and supernatant were combined, and mixed with a macroporous adsorption resin (LX-100B) in a weight ratio of 1:5, and loaded to a chromatography column. The chromatography column was eluted with 5 times the volume of water and 8 time the volume of 80% ethanol at a speed of 2 BV/h. The ethanol eluent was collected, concentrated, combined with the precipitate 2, and dried to 18.51 g of Paris polyphylla saponin extract.

### 2. Content Determination

The contents of polyphyllin II, polyphyllin VI and polyphyllin VII in the Paris polyphylla saponin extracts from Examples 5-8 were determined by HPLC, octadecylsilane-bonded silica gel as filler; acetonitrile as mobile phase A, water as mobile phase B, and gradient elution as specified in table 1, the detection wavelength: 203 nm, injection volume: 10 μL, and the column temperature: 30° C. The elution gradient is shown in Table 1.

TABLE 1
Elution Gradient for Determining the Contents
of Paris Polyphylla Saponin Extract

| Time (min) | Mobile Phase A | Mobile Phase B |
|---|---|---|
| 0-14 | 30→60 | 70→40 |
| 40-45 | 60→30 | 40→70 |

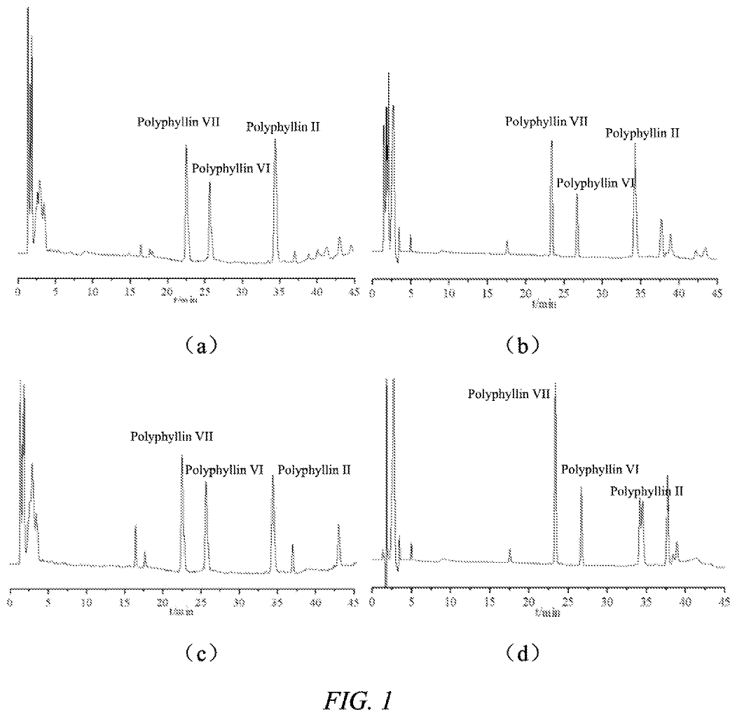

The contents of polyphyllin II, polyphyllin VI and polyphyllin VII in the Paris polyphylla saponin extracts from Examples 5-8 are shown in FIG. 1 and Table 2.

TABLE 2
Contents of Paris Polyphylla Saponin Extract

Polyphyllin content (%)

| | Polyphyllin II | Polyphyllin VI | Polyphyllin VII |
|---|---|---|---|
| Example 5 | 0.12 | 0.06 | 0.11 |
| Example 6 | 0.15 | 0.07 | 0.15 |
| Example 7 | 0.13 | 0.09 | 0.12 |
| Example 8 | 0.14 | 0.11 | 0.14 |

One gram of Paris Polyphylla Saponin Extract of Example 1 was purified by a preparative liquid phase chromatography system (Hanbang Science and Technology laboratory-grade preparative liquid chromatography system NS4210). Conditions: the chromatographic column: Hedera ODS-2 (250 mm*20 mm, 10 μm), the mobile phase: methanol-water (1-30 min, methanol 40%-70%, 30-50 min, 70%-40%), flow rate: 6 mL/min, Polyphyllin II (275.32 μg), Polyphyllin VI (61.17 μg) and Polyphyllin VII (148.85 μg) were obtained.

### 3. Evaluation of the Paris polyphylla Saponin Extract

(1) Determination of Antibacterial Activity of Paris polyphylla saponin extract by a minimum inhibitory concentration method

The minimum inhibitory concentration (MIC) of the extract was determined by liquid microdilution method.

Disinfection of Laboratory Equipment

Before the experiment, Petri dishes, test tubes, pipettes, prepared culture medium, etc. used in the experiment were autoclaved in a sterilizing pot at 121° C. for 15 min. Before the experiment, the microbiological laboratory table and 96-well plate were sterilized by ultraviolet irradiation, and the purification table was wiped with 75% alcohol.

(2) Growth Medium

18 g of dry growth medium powder was placed into a conical flask, 1000 mL of water was added. The mixture was placed in a constant temperature water bath to be heated and dissolved, and was then autoclaved at 121° C. for 20 min.

(3) Preparation of Bacterial Suspension

Twenty-four hours before the experiment, the test strains preserved in low temperature were taken out and placed at room temperature. A small amount of bacterial liquid was drawn with a pipette, inoculated into the sterilized and cooled growth medium, and placed in a 35° C. incubator for 24 hours. A pipette was used to transfer a small amount of activated bacterial colonies to a sterilized dry test tube, diluted with medium to a turbidity similar to that of a 0.5 McFarland turbidimetric tube (1.5×10⁸ CFU/mL), and then diluted with medium by 100 times, so that the bacterial suspension concentration reaches 1.0×10⁶ CFU/mL (diluted twice before being added to the wells, and the final bacterial concentration in the system being 5×10⁵ CFU/mL) for MIC measurement.

(4) Determination of Minimum Inhibitory Concentration (MIC) by Micro-Dilution Method

In a sterile 96-well flat-bottom microculture plate, 100 μL of 128 m/mL positive control solution was added to the No. 1 well of each row as a positive control; 100 μL of 128 μg/mL, 64 μg/mL, 32 μg/mL, 16 μg/mL, 8 μg/mL, 4 μg/mL, 2 μg/mL, 1 μg/mL, 0.5 μg/mL, and 0.25 μg/mL of the Paris Polyphylla Saponin Extract solutions were added to Nos. 2-11 well, respectively. No. 12 well was solvent.

The plate was incubated in a 30° C. incubator for 24 hours, the experimental results were observed, and the minimum inhibitory concentration (MIC) values were determined. When the MIC value is higher than the highest concentration of 256 m/ml, it is counted as “>256 μg/mL”; when the MIC value is the lowest concentration or below the lowest concentration, it is not counted. The difference was counted as “≤0.5 m/mL.” The above experiments were performed in parallel for 3 times. When the MIC value could be accurately repeated or only differed by one concentration, it was accepted. The higher concentration was used as the MIC value. When the MIC value differed by more than two concentrations, the experiment was repeated until it met the until requested.

The results are shown in Tables 3, 4, 5, 6, 7, 8, and 9.

TABLE 3
Tested Strains, Corresponding Positive Controls and Their Concentrations

| | | | Multidrug-resistant | Multidrug-resistant |
|---|---|---|---|---|
| | | | Staphylococcus | Pseudomonas |
| Strains | Escherichia coli | Salmonella | aureus 222 | aeruginosa 174 |
| Positive controls | Ceftriaxone sodium | Levofloxacin hydrochloride | Cefazolin sodium | Gentamicin sulfate |
| Conc. μg/mL | 128 | 128 | 128 | 128 |

TABLE 4
Results of In Vitro Antibacterial Activity
of the Paris Polyphylla Saponin Extract

| | | | Multidrug- | Multidrug- |
|---|---|---|---|---|
| | | | resistant | resistant |
| | | | Staphylococcus | Pseudomonas |
| | Escherichia | | aureus | aeruginosa |
| | coli | Salmonella | 222 | 174 |
| Positive | − | − | − | − |
| controls | | | | |
| 1024 | − | − | − | − |
| 512 | − | − | − | − |
| 256 | − | − | − | − |
| 128 | − | + | − | + |
| 64 | + | + | + | + |
| 32 | + | + | + | + |
| 16 | + | + | + | + |
| 8 | + | + | + | + |
| 4 | + | + | + | + |
| 2 | + | + | + | + |
| 1 | + | + | + | + |
| 0.5 | + | + | + | + |
| 0.25 | + | + | + | + |
| Negative | + | + | + | + |
| controls | | | | |

Note:
“+” means bacterial growth, “−” means no bacterial growth.

TABLE 5
Paris Polyphylla Saponin Extract MIC

| | | | Multidrug-resistant | Multidrug-resistant |
|---|---|---|---|---|
| | | | Staphylococcus | Pseudomonas |
| | Escherichia coli | Salmonella | aureus 222 | aeruginosa 174 |
| Paris Polyphylla | 128 | 256 | 128 | 256 |
| Saponin Extract MIC | | | | |
| (μg/mL) | | | | |

TABLE 6
Results of In Vitro Antibacterial Activity of Polyphyllin II

| | | | Multidrug-resistant |
|---|---|---|---|
| | Escherichia | | Staphylococcus |
| | coli | Salmonella | aureus 222 |
| Positive | − | − | − |
| controls | | | |
| 1024 | + | + | + |
| 512 | + | + | + |
| 256 | + | + | + |
| 128 | + | + | + |
| 64 | + | + | + |
| 32 | + | + | + |
| 16 | + | + | + |
| 8 | + | + | + |
| 4 | + | + | + |
| 2 | + | + | + |
| 1 | + | + | + |
| 0.5 | + | + | + |
| 0.25 | + | + | + |
| Negative | + | + | + |
| controls | | | |

Note:
Polyphyllin II was purified in the experiment above; “+” means bacterial growth, “−” means no bacterial growth.

TABLE 7
Results of In Vitro Antibacterial Activity of Polyphyllin VI

| | Conc. (μg/mL) | | | | | | | | | | | |
|---|---|---|---|---|---|---|---|---|---|---|---|---|
| | Positive | | | | | | | | | | | Negative |
| | Control | 128 | 64 | 32 | 16 | 8 | 4 | 2 | 1 | 0.5 | 0.25 | Control |
| Escherichia coli | − | + | + | + | + | + | + | + | + | + | + | + |
| Salmonella | − | + | + | + | + | + | + | + | + | + | + | + |

Note:
Polyphyllin VI was purified in the experiment above; “+” means bacterial growth, “−” means no bacterial growth.

TABLE 8
Results of In Vitro Antibacterial Activity of Polyphyllin VII

| | | | Multidrug- | Multidrug- |
|---|---|---|---|---|
| | | | resistant | resistant |
| | | | Staphylococcus | Pseudomonas |
| | Escherichia | | aureus | aeruginosa |
| | coli | Salmonella | 222 | 174 |
| Positive | − | − | − | − |
| controls | | | | |
| 1024 | + | + | + | + |
| 512 | + | + | + | + |
| 256 | + | + | + | + |
| 128 | + | + | + | + |
| 64 | + | + | + | + |
| 32 | + | + | + | + |
| 16 | + | + | + | + |
| 8 | + | + | + | + |
| 4 | + | + | + | + |
| 2 | + | + | + | + |
| 1 | + | + | + | + |
| 0.5 | + | + | + | + |
| 0.25 | + | + | + | + |
| Negative | + | + | + | + |
| controls | | | | |

Note:
Polyphyllin VII was purified in the experiment above; “+” means bacterial growth, “−” means no bacterial growth.

TABLE 9
Results of In Vitro Antibacterial Activity of the Combinations of
the Paris Polyphylla Saponin Extract and Antibacterial Drugs

| | Paris Polyphylla Saponin | | | Combination | | | |
|---|---|---|---|---|---|---|---|
| | Extract MIC | | | Treatment MIC | | | |
| | Single | Combination | | Single | Combination | | |
| Strains | (μg/mL) | (μg/mL) | Drugs | (μg/mL) | (μg/mL) | FIC | Results |
| Escherichia coli | 128 | 32 | Ceftriaxone | 2 | 0.5 | 0.5 | Synergy |
| | | | sodium | | | | |
| Salmonella | 256 | 128 | Levofloxacin | 4 | 2 | 1 | Addition |
| | | | hydrochloride | | | | |
| Multidrug-resistant | 128 | 128 | Cefazolin | 128 | 16 | 1.125 | No effect |
| Staphylococcus | | | sodium | | | | |
| aureus 222 | | | | | | | |
| Multidrug-resistant | 256 | 32 | Gentamicin | 0.125 | 0.125 | 1.125 | No effect |
| Pseudomonas | | | sulfate | | | | |
| aeruginosa 174 | | | | | | | |

FIC: fractional inhibitory concentration

The Paris Polyphylla saponin extract of the present invention has inhibitory activity against Escherichia coli, Salmonella, multidrug-resistant Staphylococcus aureus 222 and multidrug-resistant Pseudomonas aeruginosa 174. The MIC values of polyphyllin II, polyphyllin VI and polyphyllin VII to the experimental strains were also determined. All of which were greater than 128 μg/mL, and did not exert a good antibacterial effect. This is due to the synergistic antibacterial activity of polyphyllin II, polyphyllin VI and polyphyllin VII. The MIC values of the combinations of the Paris Polyphylla saponin extract and the positive controls (ceftriaxone sodium, levofloxacin hydrochloride, cefazolin sodium, and gentamicin sulfate) were also determined. The experimental results show that the combination of the Paris Polyphylla saponin extract and ceftriaxone sodium has a synergistic effect on the inhibition of Escherichia coli; and the combination with levofloxacin hydrochloride has an additive effect on the inhibition of Salmonella.

The above content is only to illustrate the technical idea of the present invention, and cannot limit the protection scope of the present invention. Any changes made on the basis of the technical solution according to the technical idea proposed by the present invention all fall within the scope of the claims of the present invention.

1. A method of preparing a Paris polyphylla saponin extract comprising:
(1) adding Paris polyphylla saponin in 60-90% ethanol to obtain a mixture, heating the mixture under reflux for 2-4 hours;
(2) concentrating the mixture to ¼ volume, centrifuging the mixture to obtain a supernatant A and a precipitate A, adding the precipitate A to 60-80% ethanol to obtain a supernatant B and a precipitate B, washing the precipitate B with water and petroleum ether and drying; and
(3) combining the supernatant A and the supernatant B, mixing with a macroporous adsorption resin, loading to a chromatography column, eluting the chromatography column with water and 80-90% ethanol, collecting an ethanol eluent, concentrating the ethanol eluent to obtain an eluent extract, and combining the eluent extract and the precipitate B and drying to obtain the Paris polyphylla saponin extract.

2. The method of claim 1, wherein in step (1), a weight ratio of the Paris polyphylla saponin and 60-90% ethanol is 1:8-24.

3. The method of claim 1, wherein in step (2), a weight ratio of the precipitate A and 60-80% ethanol is 1:4-10.

4. The method of claim 1, wherein the Paris polyphylla saponin extract includes 0.12%-0.14% of polyphyllin II, 0.06%-0.11% of polyphyllin VI and 0.11%-0.15% of polyphyllin VII.
